(19) United States
(12) Patent Application Publication
(10) Pub. No.: US 2019/0117262 A1
(43) Pub. Date: Apr. 25, 2019

(54) Dermatology Tool for Extraction of Sebaceous Material from Pores

(71) Applicant: Naomi Jessica Travers, Poway, CA (US)

(72) Inventor: Naomi Jessica Travers, Poway, CA (US)

(21) Appl. No.: 16/006,755

(22) Filed: Jun. 12, 2018

Related U.S. Application Data
(60) Provisional application No. 62/518,353, filed on Jun. 12, 2017.

(51) Int. Cl. A61B 17/50

(52) U.S. Cl. CPC A61B 17/50; A61B 2017/00747

(57) ABSTRACT

A lever that is designed to be used in tandem to express unwanted sebaceous material from skin pores. The levers consists of stick-like tools which are held between the thumb and forefinger and are used to exert pressure at opposing sides of the base of enlarged skin pores to expel the contents easily. The ends of each lever are of a purposeful shape to optimally expel the contents of an enlarged pore while inflicting minimal trauma to the surrounding tissue.

### FIELD OF THE INVENTION

The invention relates generally to the field of dermatology and more specifically, to the field of skin care maintenance in the form of a novel means of expressing unwanted sebaceous material from pores.

### BACKGROUND OF THE INVENTION

Acne is arguably the most common skin concern in the field of dermatology, particularly among the teenage population but also among women and, to a lesser degree, men of all ages. The precursor of many acne eruptions is a comedone, the collection of sebaceous material in a skin pore, which results in unsightly open and closed papules on the skin surface. This material, when colonized by bacteria, can lead to inflamed, red and painful pustules and cysts.

The resulting lesions are undesirable, both visually and tactilely. They are psychologically undesirable. As such, many individuals are compelled to attempt to squeeze or otherwise manipulate the lesions in order to expel the unwanted contents. This is also performed by esthetic and dermatology professionals as a means of returning the pore to its unencumbered state.

The ideal method of extruding the material from clogged pores is to exert pressure at the base from opposing sides of the lesion simultaneously. This compels the contents to exit the open pore, which is the path of least resistance. A clogged pore is typically 1 mm in diameter when distended with sebaceous material. Individuals intuitively attempt this with any number of means, including their own fingers, fingernails, cotton swabs, and other such probes.

These makeshift means are not optimal for expelling the contents of the pore. If the contact edges of the device are too acute, such as those of a fingernail, pressure is overly concentrated over a small surface area and results in trauma to the surrounding skin. If the instrument edge is too obtuse, the pressure is exerted over a surface area that is too large to deliver precise compression at the lesion base. Fingertips are generally too wide to deliver precise compression at the lesion base, while cotton swabs are generally too narrow to deliver enough pressure around or along the comedone. Cotton swabs are also dysfunctional for additional reasons. The fibers of the rough cotton surface can abrade the epidermis when pressed against the skin with enough pressure to expel pore contents. The thin lever of cotton swab, while suitable for cleaning the ear canal, is not ergonomically adapted for holding between the thumb and forefinger and often breaks under the pressure exerted when compressing the pore.

The current invention improves upon these common methods by virtue of its multiple variations of purposefully designed terminal appendages which are of an optimal size and shape for compressing human skin pores. The levers provide maximal access to the varied contours of the human face. In certain embodiments the ends of the levers where they make contact with the skin around the pore are provided with a material that is soft and malleable to protect the skin. The middle portions of the levers can also be provided with a malleable material to help the user grip the levers.

The prior art generally falls into four categories. In the first are levers with some variation of a loop or fenestrated cup at one or both ends. By far the most universally used and commercialized device is commonly known as a comedone extractor which was described at least as early as 1908 in U.S. Pat. No. 906,085, granted to Tolman. This tool is an elongated lever with loop appendages at each end. The single instrument is meant to be used by placing the loop around the lesion and pressing downward. While this method can be somewhat successful in certain circumstances, the design is inefficient because downward pressure often only serves to compress the pore content rather than expel it outward. The tool may be used to exert pressure at an angle, but this often results in shifting the pore contents to the side.

The majority of prior art for comedone extraction tools mimic the aforementioned embodiment, with only minor variations in the terminal edges and/or the type of material used. They all share the same fundamental function as a singular instrument, and thus provide suboptimal utility. U.S. Pat. No. 5,649,942 granted to Yeh virtually mimics its longstanding precursor and thus shares its limitations. Similarly, US Patent Application 2006/0155306A1 applied for by Kang, U.S. Pat. No. 5,910,147 granted to Rosenberg et al, U.S. Design Pat. 279,405 granted to Lentz, U.S. Design Pat. 212,868 granted to Olson, and U.S. Pat. No. 5,395,380 granted to Berkovich incorporate a loop appendage on only one end of a single lever.

The second category of prior art consists generally of instruments with two levers that rotate around a central axis and compress the pore from opposite sides. The benefit of simultaneous pressure from the two levers on both sides of the pore is negated by their connection at a single point, which limits their mobility to one plane.

For example, U.S. Pat. No. 783,924 granted to Boehm and U.S. Pat. No. 896,338 granted to Tolman are similar embodiments which resemble common tweezers. U.S. Pat. No. 896,338 and US Patent Application 2010/0185214A1 applied for by Lonn are variations of this general design with a U-shaped arch acting as the juncture of the two compressing levers. U.S. Pat. No. 5,431,665 granted to Li and U.S. Pat. No. 834,683 granted to Severin are similar embodiments that have a looped appendage at the juncture of two levers forming a tweezer-like tool. Other embodiments such as US Patent Application 2013/0144308A1 applied for by Forrest employ two levers which rotate around a central axis such as a pair of scissors. Although these embodiments allow for simultaneous compression at the pore base, which is ideal for extraction, the attachment of the two levers constrains the overall utility of these tools.

The third category includes miscellaneous embodiments of singular tools with various means of extracting sebaceous material. U.S. Pat. No. 2,267,636 granted to Benton utilizes a spring-loaded plunger mechanism to compress the skin around the pore. The tool is placed directly over the pore, thus preventing the user from visualizing the extraction site. U.S. Pat. No. 6,019,749 granted to Fields et al is a relatively complex design that employs suction to extract the pore contents and shares the same limitations as U.S. Pat. No. 2,267,636. The complexity of the design would make it impractically expensive to manufacture these designs.

There fourth category of devices are similar to the present invention where two separate members are used to apply pressure to the comedone from opposite sides as discussed above with respect to cotton swabs. US Patent Application 2010/0185214A1 applied for by Lonn, discussed above, also teaches a device where two separate levers are used. The Lonn application teaches levers with round, bulbous ends. US Patent Application 2011/0087239A1 applied for by Howell consists of two levers that are meant to be secured to each forefinger. This securement to the forefingers by the use of adhesive means their utility is limited to the dexterity of those digits. Also it seems that if the adhesive is strong enough to hold the levers onto the fingers, it would also presumably cause trauma upon removal from the skin.

There is a need in the field for a comedone extraction device that uses two levers that are held in each hand with ends having a particular form that can also be held together during storage. The current invention provides such a device.

### SUMMARY OF THE INVENTION

The current invention consists of a lever which can be used in pairs. The ends of the lever are of a particular form. The levers can be provided with similarly shaped but differently dimensioned ends or tips. The ends can be flattened or sloped toward the ends on opposing top and bottom surfaces. The ends of the levers are generally wider than they are high. The opposed bottom and top surfaces are preferably similarly shaped and mirror each other through a plane through the elongated axis of the lever. The opposed left and right side surfaces are also similarly shaped and mirror each other through a plane through the elongated axis of the lever. The junctures of the surfaces of the ends or tips are also rounded. The ends of the levers are preferably solid. The user preferably holds one of each lever in the right and left hands and places the chosen end of each lever at the base of or adjacent the lesion of the targeted pore with the ends of the tools in opposed relation on either side of the pore. By exerting pressure at the base of the lesion from opposing sides, the contents are naturally compelled to exit via the path of least resistance, i.e. the pore opening. The current invention describes two levers which are held by the thumb and forefinger, which affords superior dexterity and precision in the placement of the levers. Also the pore opening may be easily visualized between the instrument ends.

The opposing tips or opposing ends of each lever can be the same or distinct shapes. Preferably, the levers are elongated, straight members with opposed ends. The opposing ends can also vary in size to accommodate different comedone sizes. The ends of the levers are generally narrower than the middle of the levers. Preferably, the levers are larger in their middle sections for ease of grasping between the thumb and forefinger, and narrower at their ends to limit the surface area contact they make with the skin around the pore. The ends of the levers can be shaped like a common spatula which is to say the bottom and top surfaces of the ends can be flattened, compressed or indented such that the opposed top and bottom surfaces are generally parallel to each other to create a more narrow surface area of contact. Similarly, the top and bottom surfaces can slope towards the ends to make a narrower end surface for the lever. The ends of the levers are formed with an end surface or terminal end and left and right side surfaces surface between the top and bottom surfaces of the lever. All of the surfaces can be rounded, somewhat rounded or flat, and the edges between the surfaces are rounded so that the levers lack sharp edges between the surfaces so as to prevent trauma to the skin. The left and right side surfaces can be flattened near the end to narrow the end of the lever or they can slope toward the end to narrow the end surface. The end surface and the left and right side surfaces can form a singular curving surface or they can be multiple surfaces with rounded junctures between them. Similarly, the end surface and the top and bottom surfaces can form a singular curving surface or they can be multiple surfaces with rounded junctures or edges between them. All junctures between surfaces are rounded so that no sharp edges or junctures are provided. The ends of the levers provide a smooth surface where they make contact with the skin. This contact surface has no sharp edges. Preferably, the ends of the lever are of different shape and sizes to accommodate pores of different sizes and enlargement. In one embodiment, one end is more bulbous and more blunt than the other.

The ends are preferably between approximately 1 and 6 millimetres wide and approximately 0.05 to 2 mm thick or high. The edges or junctures between the surfaces along the width, height and depth are rounded in order to minimize trauma to the skin. The levers are preferably approximately 10 to 15 cm long. The middle portion of the levers can have a rounded cross-section or any other cross-section that will help the levers be gripped by the fingers. The middle portion of the levers can be approximately 1 cm to 2 cm in diameter. One of the tips can have an end surface that is generally flattened approximately 4 mm wide and 1 mm high. The other of the tips can have an end surface that is very rounded with the width dimension between the left and right side surfaces being approximately 5 mm and the height of the end surface at its largest height being approximately 2 mm in height.

The tips or ends of the levers can be provided with a soft malleable material where the ends of the lever make contact with the skin around the pores. This material can be silicone or a similarly soft, malleable material to minimize trauma. Silicone rubber is also easily cleaned for reusing the lever. The tips are preferably at a 180 degree angle with the handle or middle portion of the levers, but the tips or ends may also be disposed at a lesser angle. The tips or ends can be joined to the handle portions of the levers by neck portions that are thinner than either the ends or the handle portions of the levers. The levers can be provided with one or more reinforcing members within the lever to strengthen the levers, especially at the narrow neck portions of the levers near the ends.

For convenient storage of the two levers, it is desirable to have couplers for securing them to each other. The preferable method of attachment is to use magnetic attraction between the levers. The handles or cores or portions of these members of the levers may be made from magnetically attractive materials or alternatively, magnetically attractive materials of any shape or form may be embedded within the levers. Preferably, the magnetically attractive materials are disposed far enough from the ends or are weak enough that the user does not notice any magnetic attraction between the levers when using them.

Alternatively, the two levers may each accommodate any variation of conforming male and female structures or other interlocking strictures which can be easily coupled to secure the levers for storage and uncoupled to separate them for use. The mechanism could be notches in the tools that snap fit together.

The present invention may be made entirely of one single material, or a combination of materials. In one example, the handle or middle portion of the levers can be made from one material and the terminal ends where contact is made with the skin can be made of a second material. The levers are preferably strong and firm enough to translate force from the hands to the ends of the levers. In another example, the levers are made with a rigid core that is encased in a softer, malleable material, especially at the ends where contact is made with the skin. In one embodiment the core is made from hard plastic and is completely surrounded by a soft, malleable outer later that is at a minimum 1 mm thick. The outer later can be as thick as 3 to 4 mm, although as the outer layer becomes thicker a firmer silicone rubber or other material may need to be used to make the ends of the device firm enough to exert sufficient pressure on the skin surrounding the comedone. The inner core is generally shaped similarly to the outer surface of the lever when the malleable outer layer or casement is applied to the inner core.

The handle or middle portion of the levers or the core of the levers or the entire levers may be made from any number of firm materials such as stainless steel, acrylic, plastic, wood, or any derivatives of these materials. Various materials and construction of the handles and tips are dictated by the overriding goal of making the tools functional, stable and reusable. The material can be non-corrosive.

The current invention affords a level of precision not previously taught by existing prior art in the field, the vast majority of which consist of singular instruments manipulated by one hand. The use of two separate levers allows optimal utility for several reasons; first, the two levers can be placed at opposing sides of the base of the filled pore and used to exert pressure simultaneously. This is the most efficient means of expelling the contents. The user may push on the skin near or adjacent the comedone with the end surfaces of the levers or the left and right side surfaces of the ends of levers. The elongated axis of the levers can be set at angle to the face or they can be parallel with the surface of the face where the comedone exits.

Secondly, the functionality of two separate levers is superior to any single tool because they allow the user to recruit the joints of the thumb, forefinger and wrists of both hands, which maximizes the number of positional permutations available to the user. This increases access to the complex and varied contours of the human face where comedones may exist.

### BRIEF DESCRIPTION OF THE DRAWINGS

FIG. 1 is top view of two levers of the present invention.

FIG. 2 is a side view of a lever of the present invention.

FIG. 3 is a top view of the lever of FIG. 2.

FIG. 4 is cut-away perspective view of the lever of FIG. 2.

FIG. 5 is a perspective view of the lever of FIG. 2.

FIG. 6 is an end view of the lever of the present invention taken along line FIG. 6 of FIG. 1.

FIG. 7 is an end view of the lever of the present invention taken along line FIG. 7 of FIG. 1.

FIG. 8 is a top view of two levers joined to each other by magnets embedded in the levers.

FIG. 9 shows the levers being used.

FIG. 10 is a top view of two levers to each other by couplers.

FIG. 11 is an enlarged top view of one end of the lever taken along line FIG. 11 of FIG. 1.

FIG. 12 is an enlarged top view of one end of the lever taken along line FIG. 12 of FIG. 1.

FIG. 13 is an enlarged side view of one end of the lever taken along line FIG. 13 of FIG. 2.

FIG. 14 is an enlarged side view of one end of the lever taken along line FIG. 14 of FIG. 2.

### DETAILED DESCRIPTION OF THE INVENTION

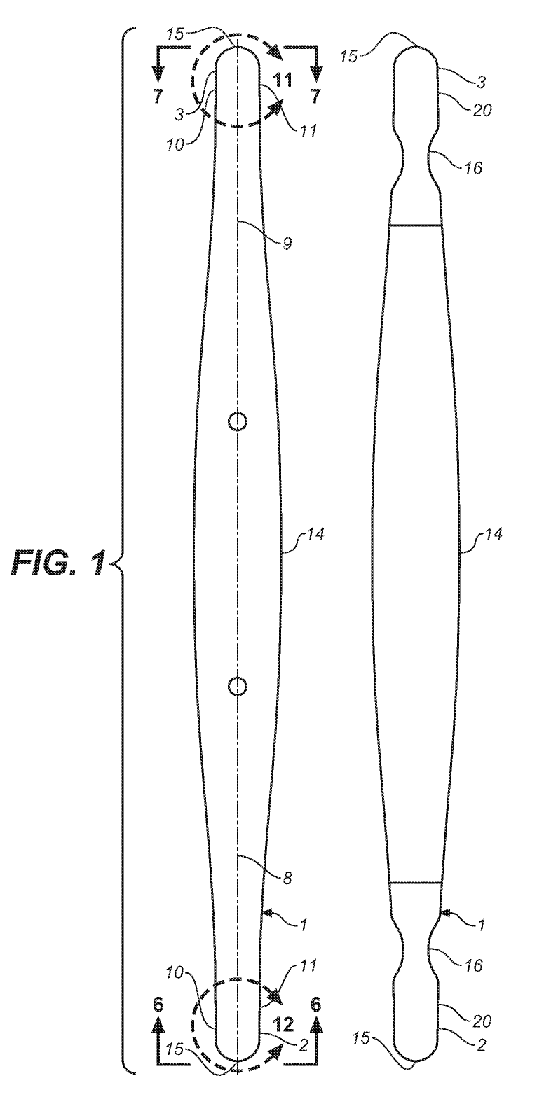

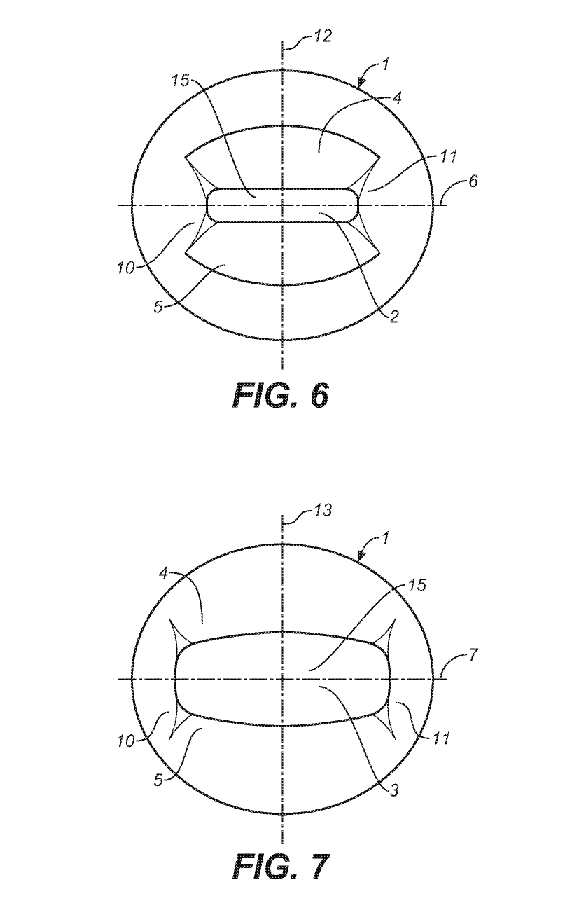

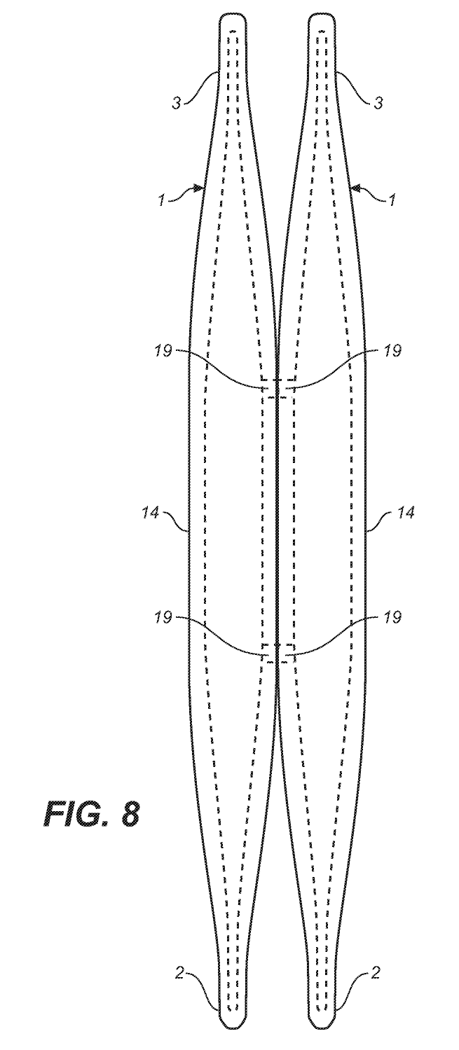

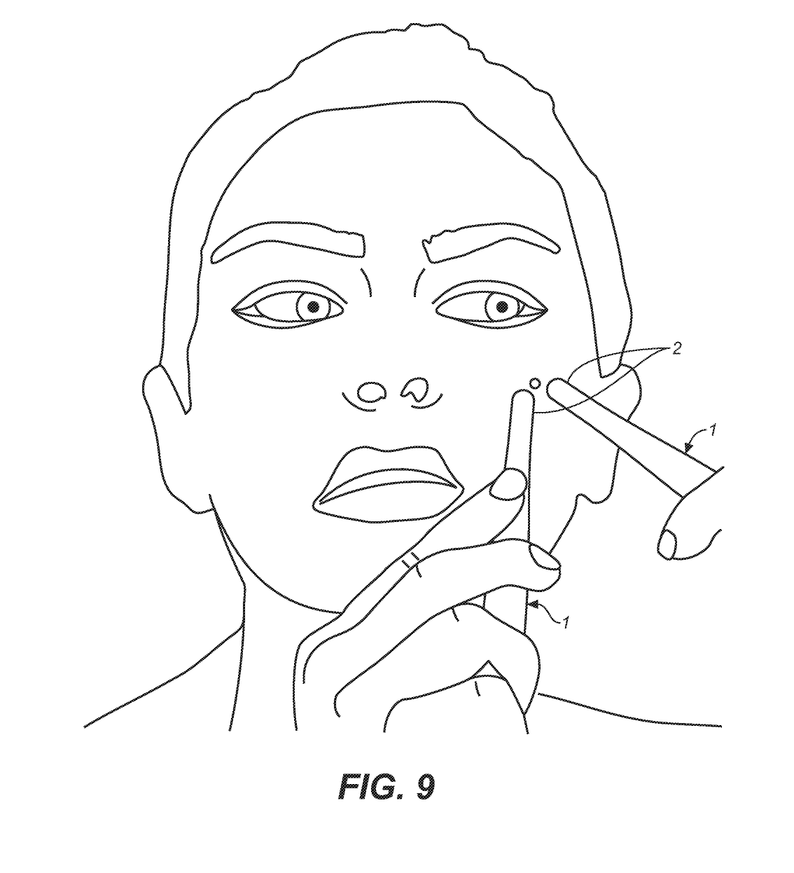

As shown in FIG. 1, the current invention consists of a lever 1 which can be used in pairs. The ends 2 and 3 of the lever 1 are of a particular form. The levers 1 can be provided with similarly shaped but differently dimensioned ends or tips 2 and 3. The ends 2 and 3 can be flattened or sloped toward the ends on opposing top and bottom surfaces 4 and 5. As shown in FIGS. 6 and 7, the opposed top and bottom surfaces 4 and 5 are preferably similarly shaped and mirror each other through a plane 6 or 7 through an elongated axis 8 or 9 of the first or second end 2 and 3 of the lever 1. The planes 6 and 7 and elongated axis 8 and 9 in the preferred embodiment shown in the drawings are aligned. The opposed left and right side surfaces 10 and 11 are also preferably similarly shaped and mirror each other through a plane 12 or 13 through the elongated axis 8 or 9 of the first or second end of the lever 1. As shown in FIG. 1, the elongated axes 8 and 9 in the preferred embodiment shown in the drawings are aligned. The junctures of the top and bottom surfaces 4 and 5 with the left and right side surface 10 and 11 of the ends or tips 2 and 3 are rounded. The ends 2 and 3 of the levers 1 are preferably solid. As shown in FIG. 9, the user preferably holds one of each lever 1 in the right and left hands and places the chosen end 2 or 3 of each lever 1 at the base of or adjacent the lesion of the targeted pore with the select ends 2 or 3 of the levers 1 in opposed relation on either side of the pore. By exerting pressure at the base of the lesion from opposing sides, the contents are naturally compelled to exit via the path of least resistance, i.e. the pore opening. The current invention describes two levers 1 which are held by the thumb and forefinger, which affords superior dexterity and precision in the placement of the levers 1. Also the pore opening may be easily visualized between the instrument ends 2 or 3.

The tips or ends 2 and 3 of each lever 1 can be the same or distinct shapes. As shown in FIG. 1, preferably, the levers 1 are elongated, straight members with opposed ends 2 and 3 with the ends 2 and 3 differing from each other in shape and size. The opposing ends 2 and 3 can also vary in size to accommodate different comedone sizes. The ends 2 and 3 of the levers 1 are generally narrower than the middle portion 14 of the levers 1. Preferably, the levers 1 are larger in their middle portions 14 for ease of grasping between the thumb and forefinger, and narrower at their ends 2 and 3 to limit the surface area contact they make with the skin around the pore. The ends 2 and 3 of the levers 1 can be shaped like a common spatula which is to say the bottom and top surfaces 5 and 6 of the ends 2 and 3 can be flattened, compressed or indented such that the opposed top and bottom surfaces 5 and 6 are generally parallel to each other to create a more narrow surface area of contact. Similarly, the top and bottom surfaces 5 and 6 can slope towards the ends 2 and 3 to make narrower end surfaces 15 for the lever 1. The ends 2 and 3 of the levers 1 are formed with an end surface or terminal end 15 and left and right side surfaces 10 and 11 between the top and bottom surfaces 4 and 5 of the lever 1. All of the surfaces 4, 5, 10, 11 and 15 can be rounded, somewhat rounded or flat, and the edges between the surfaces 4, 5, 10, 11 and 15 are rounded so that the levers 1 lack sharp edges between the surfaces 4, 5, 10, 11 and 15 that make contact with the skin so as to prevent trauma to the skin. The left and right side surfaces 10 and 11 can be flattened near the end to narrow the end 2 or 3 of the lever 1 or they can slope toward the end 2 or 3 to narrow the end surface 15. The end surface 15 and the left and right side surfaces 10 and 11 can form a singular curving surface or they can be multiple surfaces with rounded junctures between them. Similarly, the end surface 15 and the top and bottom surfaces 4 and 5 can form a singular curving surface or they can be multiple surfaces with rounded junctures or edges between them. All junctures between surfaces 4, 5, 10, 11 and 15 are rounded so that no sharp edges or junctures are provided. The ends 2 and 3 of the levers 1 provide a smooth surface where they make contact with the skin. This contact surface has no sharp edges. Preferably, the ends 2 and 3 of the lever are of different shape and sizes to accommodate pores of different sizes and enlargement. In one embodiment, one end is more bulbous in shape than the other while the other is more flattened.

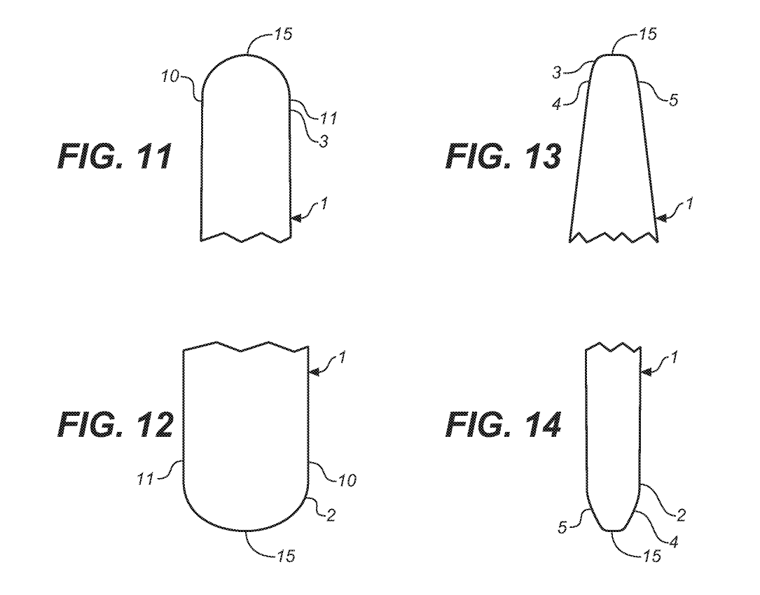

The ends 2 and 3 are preferably between approximately 1 and 6 millimetres wide between the left and right surfaces 10 and 11 and approximately 0.05 to 2 mm thick or high between the top and bottom surfaces 4 and 5. The edges or junctures between the surfaces 4, 5, 10, 11 and 15 along the width, height and depth are rounded in order to minimize trauma to the skin. The end 2 shown in FIGS. 6, 12 and 14 has an end surface 15 that is 4.0 mm wide and 1.0 mm high. The top and bottom surface 4 and 5 slope toward the end surface 15 of end 2. The top and bottom surfaces 4 and 5 extend along the lever 1 approximately 2 mm. The end 3 shown in FIGS. 7, 11 and 13 has an end surface 15 that is very curved and extends 5 mm between the left and right surfaces 10 and 11. The height of the end surface 15 of end 3 is 2 mm. The top and bottom surfaces 4 and 5 of end 3 slope toward the end surface 15 of end 3. The levers 1 are preferably approximately 10 to 15 cm long. The middle portion 14 of the levers 1 can have a rounded cross-section or any other cross-section that will help the levers be gripped by the fingers. The middle portion 14 of the levers 1 can be approximately 1 cm to 2 cm in diameter. One of the tips 2 or 3 can have an end surface 15 that is generally flattened approximately 4 mm wide and 1 mm high. The other of the tips 2 or 3 can have an end surface 15 that is very rounded with the width dimension between the left and right side surfaces 10 and 11 being approximately 5 mm and the height of the end surface 15 at its largest height between the top and bottom surfaces 4 and 5 being approximately 2 mm in height.

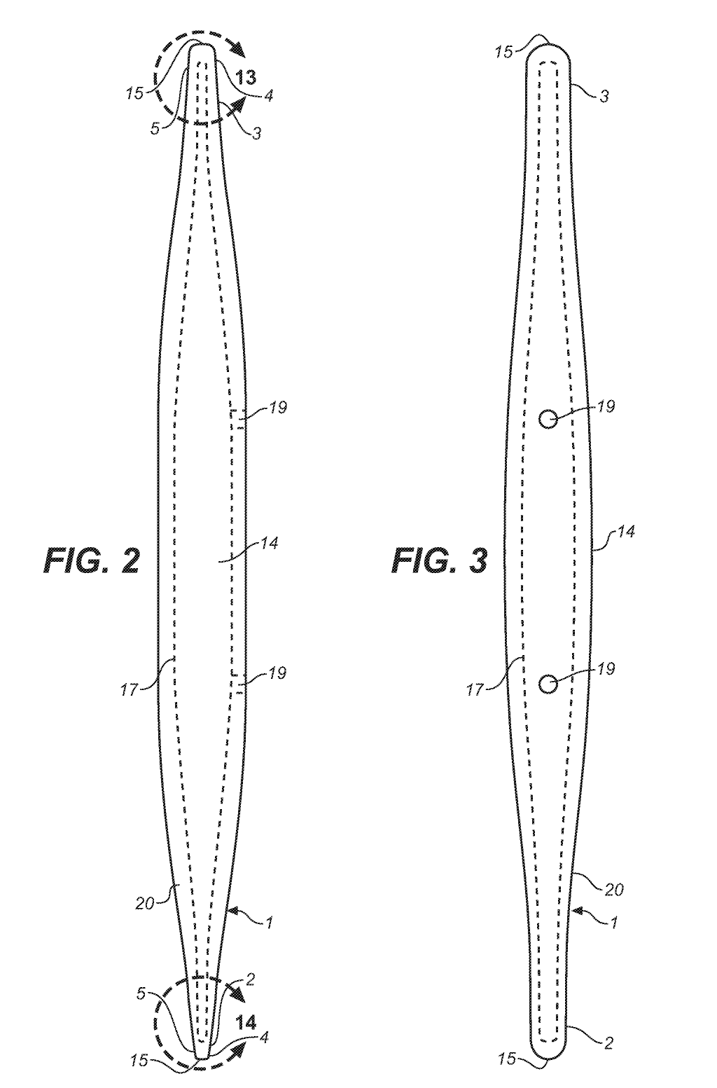

AS shown in FIG. 1 on the lower lever in the drawing 1, the tips or ends 2 and 3 of the levers can be provided with a soft malleable material 20 where the ends of the lever make contact with the skin around the pores. This material can be silicone or a similarly soft, malleable material to minimize trauma. The malleable material 20 on the ends 2 and 3 can be removable by the user. Silicone rubber is also easily cleaned for reusing the lever 1. As shown in FIGS. 1 and 2, the tips 2 and 3 are preferably at a 180 degree angle with the handle or middle portion 15 of the levers 1, but the tips or ends 2 and 3 may also be disposed at a lesser angle. As shown in FIG. 1, the tips or ends 2 and 3 can be joined to the handle or middle portions 14 of the levers 1 by neck portions 16 that are thinner than either the ends 2 o4 3 or the middle portions 14 of the levers 1. The levers 1 can be provided with one or more cores or reinforcing members 17 within the lever 1 to strengthen the levers 1, especially at the narrow neck portions 16 of the levers 1 near the ends 2 and 3.

For convenient storage of the two levers 1, it is desirable to have coupling structures or couplers 18 for securing them to each other. The preferable method of attachment is to use magnetic attraction between the levers 1. The reinforcing members 17 or portions of these members 17 of the levers 1 may be made from magnetically attractive materials or alternatively, magnetically attractive plugs 19 of any shape or form may be embedded within the levers 1 as shown in FIGS. 2 and 3. Preferably, the magnetically attractive plugs 19 are disposed far enough from the ends 2 and 3 or are weak enough that the user does not notice any magnetic attraction between the levers 1 when using them.

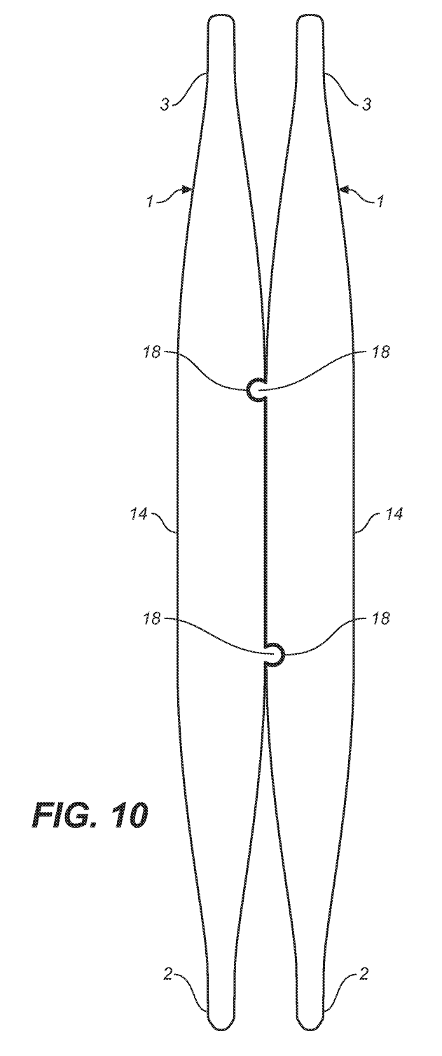

As shown in FIG. 10, alternatively, the two levers 1 may each accommodate any variation of conforming male and female coupling structures 18 or other interlocking structures which can be easily coupled to secure the levers 1 for storage and uncoupled to separate them for use. The mechanism could be notches in the levers that snap fit together.

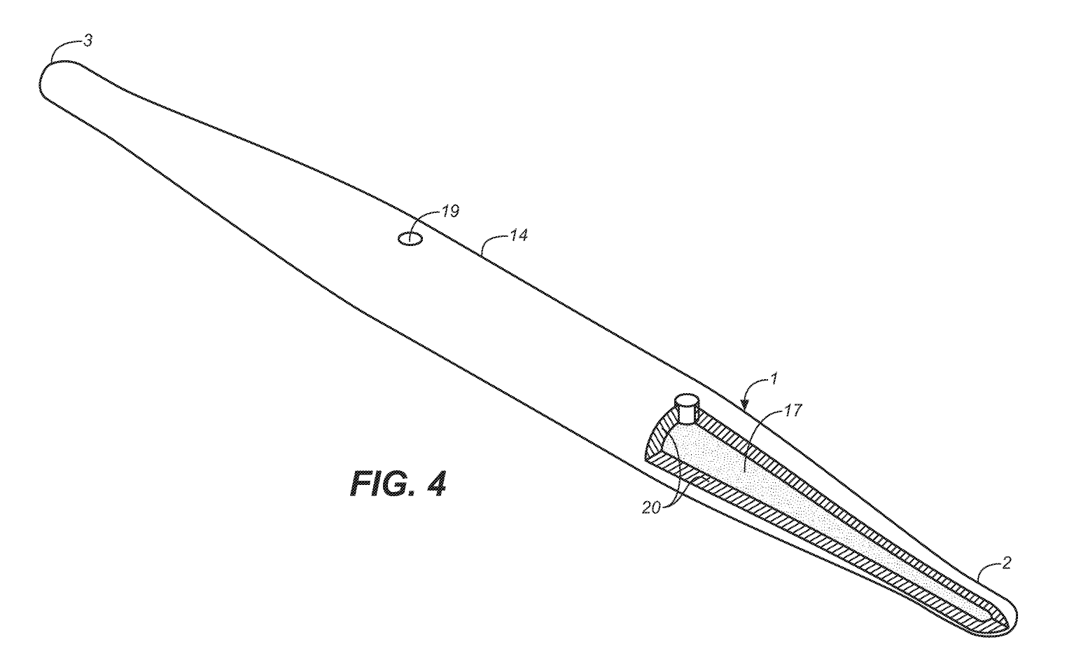

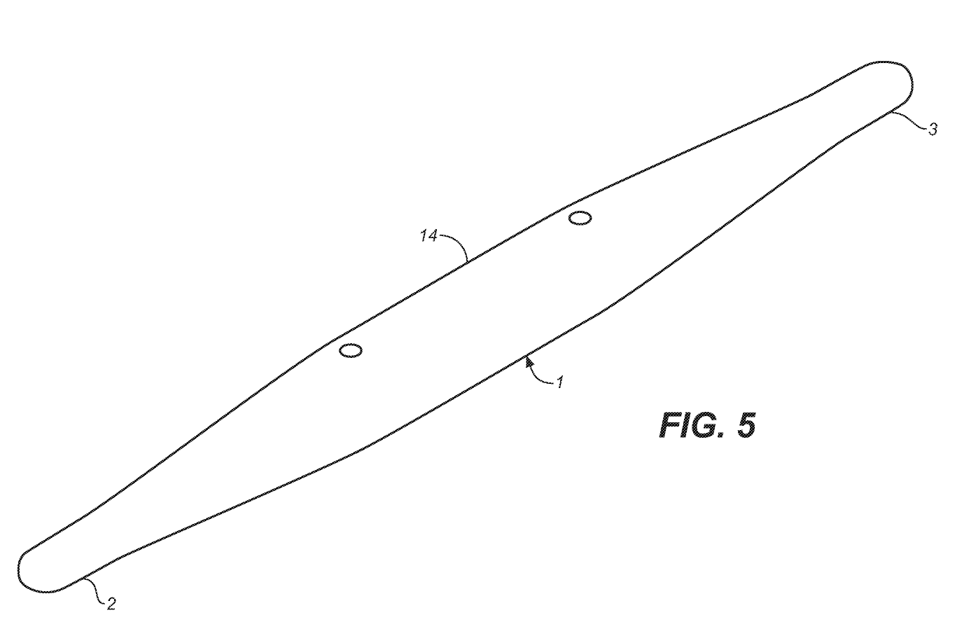

The lever 1 may be made entirely of one single material, or a combination of materials. In one example, the handle or middle portion 14 of the levers 1 can be made from one material and the terminal ends 2 and 3 where contact is made with the skin can be made of a second material or covered with a second material. The levers are preferably strong and firm enough to translate force from the hands to the ends of the levers 1. As shown in FIG. 4, in another example, the levers 1 are made with a rigid core 17 that is encased in a softer, malleable material 20, especially at the ends 2 and 3 where contact is made with the skin. In one embodiment the core 17 is made from hard plastic and is completely surrounded by a soft, malleable outer layer 20 that is at a minimum 1 mm thick. The outer layer 20 can be as thick as 3 to 4 mm, although as the outer layer 20 becomes thicker a firmer silicone rubber or other material may need to be used to make the ends 2 and 3 of the lever 1 firm enough to exert sufficient pressure on the skin surrounding the comedone. The inner core 17 is generally shaped similarly to the outer surface of the lever 1 when the malleable outer layer or casement is applied to the inner core 17.

The handle or middle portion 14 of the levers 1 or the core 17 of the levers or the entire levers 1 may be made from any number of firm materials such as stainless steel, acrylic, plastic, wood, or any derivatives of these materials. Various materials and construction of the middle portion 14 and tips 2 and 3 are dictated by the overriding goal of making the tools functional, stable and reusable. The material can be non-corrosive.

I claim:

1. A device for extracting material from a comedone, comprising:
a first lever having a middle portion with first and second ends, a first end of the first lever having top and bottom surfaces that intersect with an end surface and left and right side surfaces between the top and bottom surfaces that intersect with the end surface, and a second end of the first lever having top and bottom surfaces that intersect with an end surface and left and right side surfaces between the top and bottom surfaces that intersect with the end surface.

2. The device of claim 1, wherein
the end surface of the first end is dimensioned to be wider between the left and right side surfaces than the end surface of the first end is tall between the top and bottom surfaces.

3. The device of claim 1, wherein
the end surface of the second end is dimensioned to be wider between the left and right side surfaces than the end surface of the first end is tall between the top and bottom surfaces.

4. The device of claim 1, wherein:
the first and second ends are shaped differently.

5. The device of claim 1, wherein:
the first and second ends of the lever are encased in a soft, malleable material.

6. The device of claim 1, wherein:
the top and bottom surfaces of the first end slope toward the end surface such that the first end narrows toward the end surface.

7. The device of claim 1, wherein:
the top and bottom surfaces of the second end slope toward the end surface such that the first end narrows toward the end surface.

8. The device of claim 1, wherein:
the left and right side surfaces of the first end slope toward the end surface such that the first end narrows toward the end surface.

9. The device of claim 1, wherein:
the left and right side surfaces of the second end are generally parallel near the end surface.

10. The device of claim 1, further comprising:
a second lever having a middle portion with first and second ends, a first end of the second lever having top and bottom surfaces that intersect with an end surface and left and right side surfaces between the top and bottom surfaces that intersect with the end surface, and a second end of the first lever having top and bottom surfaces that intersect with an end surface and left and right side surfaces between the top and bottom surfaces that intersect with the end surface.

11. The device of claim 10, wherein
the end surfaces of the first ends of the first and second levers are dimensioned to be wider between the left and right side surfaces than the end surfaces of the first ends are tall between the top and bottom surfaces.

12. The device of claim 10, wherein
the end surfaces of the second ends of the first and second levers are dimensioned to be wider between the left and right side surfaces than the end surfaces of the first ends of the first and second levers are tall between the top and bottom surfaces.

13. The device of claim 10, wherein:
the first and second ends of each of the first and second levers are shaped differently.

14. The device of claim 10, wherein:
the first and second ends of the first and second levers are encased in a soft, malleable material.

15. The device of claim 10, wherein:
the top and bottom surfaces of the first ends of the first and second levers slope toward the end surfaces such that the first ends narrow toward the end surfaces.

16. The device of claim 10, wherein:
the top and bottom surfaces of the second ends of the first and second ends slope toward the end surfaces such that the first ends narrow toward the end surfaces.

17. The device of claim 10, wherein:
the left and right side surfaces of the first ends of the first and second levers slope toward the end surfaces such that the first ends narrow toward the end surfaces.

18. The device of claim 10, wherein:
the left and right side surfaces of the second ends of the first and second levers are generally parallel near the end surfaces.

19. A device for extracting material from a comedone, comprising:
a. a first lever having a middle portion with first and second ends; and
b. a second lever having a middle portion with first and second ends;
wherein
c. releasable coupling mechanisms are provided on the levers to join them together when not in use.
